(19) United States
(12) Plant Patent Application Publication
(10) Pub. No.: US 2003/0163860 P1
(43) Pub. Date: Aug. 28, 2003

(54) Kiwi plant named 'Hortgem Rua'

(76) Inventors: Mark A. McNeilage, Auckland (NZ); Ron A. Beatson, Motueka (NZ); Elspeth A. MacRae, Auckland (NZ)

Correspondence Address: GREENLEE WINNER AND SULLIVAN P C, 5370 MANHATTAN CIRCLE, SUITE 201, BOULDER, CO, 80303, US

(21) Appl. No.: 10/084,606

(22) Filed: Feb. 25, 2002

(51) Int. Cl. A01H 5/00

(52) U.S. Cl. PLT/156

(57) ABSTRACT

A new and distinct kiwi plant of the species Actinidia arguta (Sieb. & Zucc.) Planch. ex Miq. var. arguta is described. The variety results from a controlled pollination using a female A. arguta selection AAME01_01 (unpatented) and a male A. arguta selection AAME01_05 (unpatented). The fruit of this new variety has an attractive appearance (both internal and external) characterised by its green hairless, edible skin with red-centered flesh at maturity, small fruit size, oblong shape, and sweet aromatic taste. The new variety has been named ‘Hortgem Rua’.

### BACKGROUND TO THE INVENTION

Kiwi plants in cultivation are mainly varieties of A. deliciosa, particularly ‘Hayward’ although some A. chinensis and A. arguta varieties are grown. A. deliciosa and A. chinensis are closely related, whereas A. arguta is classified in a separate section of the genus. A. deliciosa and A. chinensis varieties have large fruit (˜100 g) with hair on the skin. The main varieties in New Zealand are ‘Hayward’ (A. deliciosa) and ‘Hort16A’ (A. chinensis). Fruit are usually cut and eaten with a spoon. A. Arguta has small fruit (˜10 g) with no hair on the skin. The skin is edible so these fruit can be eaten whole, like a grape.

All Actinidia species are dioecious, so female varieties have to be interplanted with male pollinizers to ensure fruit production.

A. arguta vines are deciduous and tend to grow vigorously in spring and summer when rapidly-growing shoots can intertwine and tangle if not managed. Vines do best in a mild temperate climate without late spring or early autumn frosts. They produce consistent heavy crops when grown in well-drained fertile soils and given regular irrigation in dry spells.

A. arguta flowers in spring (late October-early December) in New Zealand. Harvest of A. arguta fruit may occur between early February and late March in New Zealand depending on the selection and location of plantings. Compared to A. deliciosa and A. chinensis, A. arguta fruit require more careful handling during harvest and post-harvest procedures.

### SUMMARY OF THE INVENTION

The new variety was selected from a population of seedlings derived from crossing the A. arguta selection AAME01_{—}01 (unpatented) and a male A. arguta selection AAME01_{—}05 (unpatented). This new variety was created during the course of a planned plant-breeding program, which was initiated during 1987 at HortResearch in Auckland, New Zealand. The controlled cross was made in November 1987. Seeds were sown in autumn (March) 1988 and seedlings were selected from this cross and were planted out in the field at HortResearch Kumeu Research Orchard in spring (October) 1988. The seedlings first fruited in February-March 1991. Promising female seedlings were clonally propagated into a two-site replicated trial in 1995 and ‘Hortgem Rua’ (breeding code E4I6) was selected after storage and sensory evaluation in 1998.

The new variety can be asexually reproduced as cuttings or by grafting or budding on to seedling or cutting-grown rootstocks of A. arguta. Trial plantings as cuttings established in 1995 at the HortResearch Te Puke and Nelson Research Centres and on seedling rootstocks established in 1998 at these sites have shown that the unique combination of characters come true to form and are established and transmitted through succeeding asexual propagations.

### BRIEF DESCRIPTION OF THE ILLUSTRATIONS

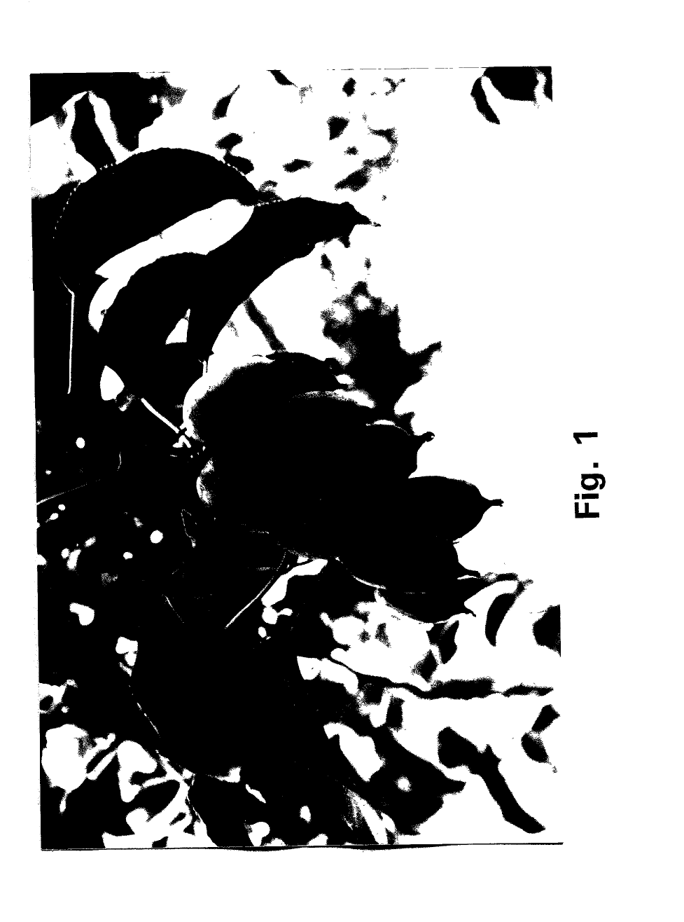

FIG. 1 shows typical fruit of the variety ‘Hortgem Rua’ in the orchard

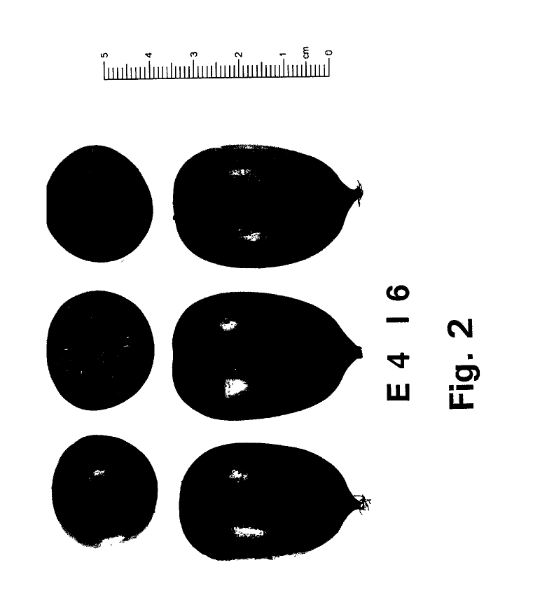

FIG. 2 shows typical fruit of the variety ‘Hortgem Rua’ in the studio

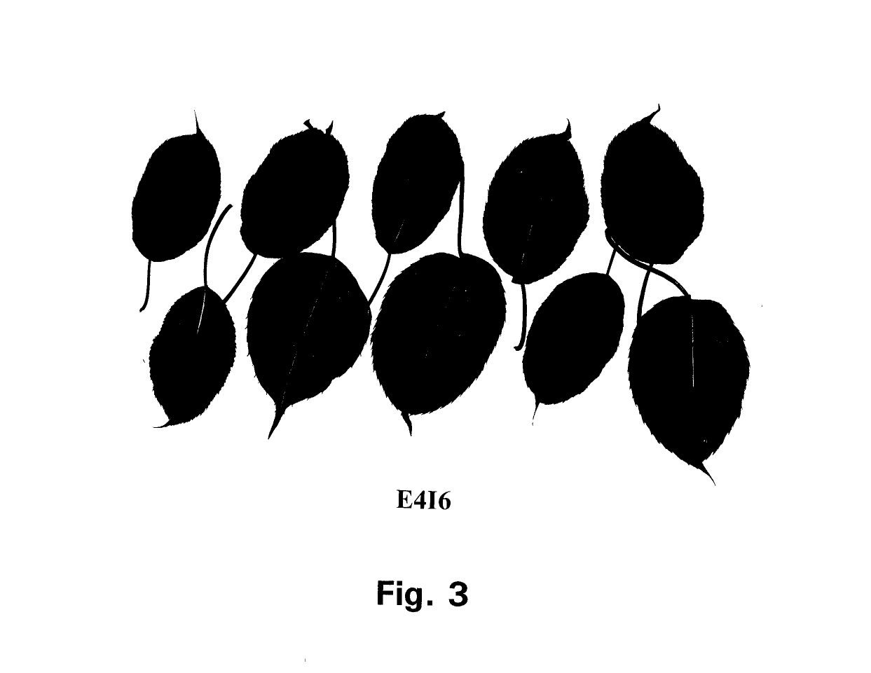

FIG. 3 shows leaves of the variety ‘Hortgem Rua’

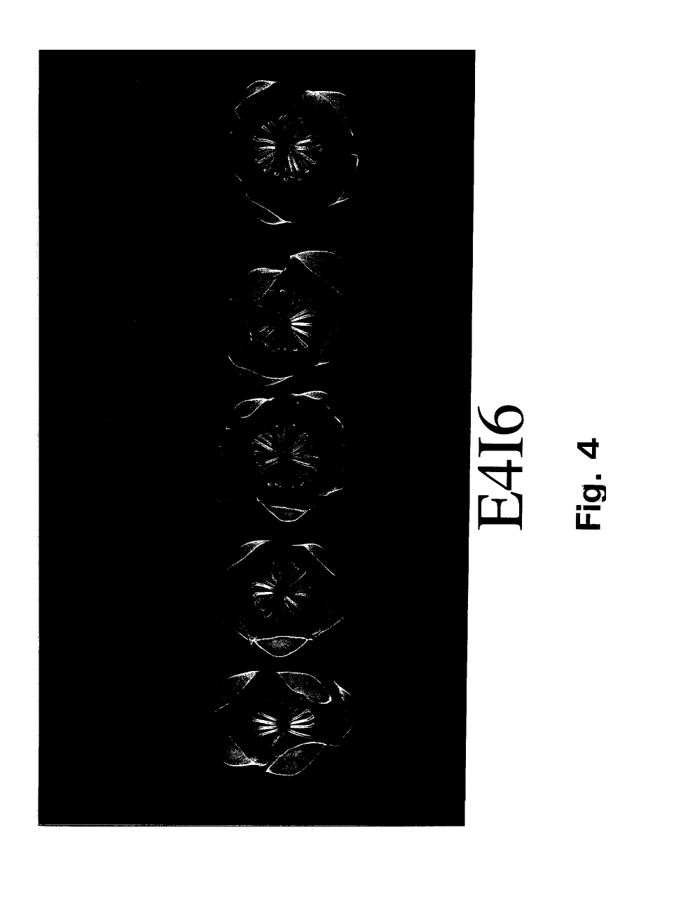

FIG. 4 shows flowers of the variety ‘Hortgem Rua’

Photographs of fruit were taken after the normal harvest date and are depicted in colours as nearly true as is reasonably possible to make the same in a colour illustration of this character. Fruit skin colour may vary depending upon extent of exposure to direct sunlight.

### MORPHOLOGICAL DESCRIPTION OF THE VARIETY

The following is a detailed description of the new variety. The specimens described were grown at HortResearch Te Puke Research Orchard, Bay of Plenty, New Zealand. The observations were made in the 1998 season on vines established in 1995 that were three years old at the time, and managed under standard orchard practice.

Plant

Character.—Medium size vine; twining habit; bearing on spurs; medium vigour.

Trunk.—Moderately rough; the bark is reddish-brown when mature.

Laterals.—Bark reddish-brown.

Lenticels.—Medium size; average numbers on current years growth.

Leaves.—Length 91 mm; width 50 mm; upward pose; upfolded to concave shape in cross section; serrate indentations of margin; medium glossiness on upper surface; petiole length 23-25 mm; medium stipule size; medium time of bud burst; colour of blade is green.

Flower buds.—Small size.

Flowers.—Medium blooming period, commencing early November and approximately 10 days in duration; predominately three flowers per inflorescence; five petals overlapping; colour green-white; generally five sepals small size, pointed, green with red tips; many stamens with white filaments and black anthers; smooth ovary.

Fruit:

Examined at optimum.—Harvest date (determined when all fruit contained black seeds and 1% of fruit on the vine were soft): 24^{th} February in Bay of Plenty, New Zealand.

Harvest.—Mid season.

Size.—Small to medium; average weight, 16.4 g (range 14-18 g).

Shape.—Generally oblong.

Skin.—Thin, smooth; free of hairiness.

Skin Colour.—(Outer pericarp colour at maturity for consumption) medium green.

Flesh.—Tender.

Flesh Colour.—Green progressing to red (red core extending into the locule area) at maturity for consumption.

Fruit colour development.—Green skin and green flesh throughout fruit growth; flesh colour change typically commences approximately 109 days after anthesis. Reddening typically occurring at the end of the fruit that softened first; from the stem end down.

Flavour.—Medium sweetness; 12.4 Brix at harvest (mean 7.4 Brix, mean maximum 15.8 Brix); aromatic.

Dry Matter Content.—Mean 18.6% (mean maximum 20.2%)

Storage.—3-5 weeks at 0° C. in air storage.

### HORTICULTURAL CHARACTERISTICS

Details below relate to observations made on cutting-grown plants growing at HortResearch Te Puke Research Centre, New Zealand. These vines were 3 years old. Data from harvesting all fruit from vines in early February 1998.

Cropping: Young vines of ‘Hortgem Rua’ are precocious, beginning to bear in their second year and are expected to reach full capacity at about 7 years. The storage life of ‘Hortgem Rua’ fruit is 3-5 weeks at 0° C., when stored in unventilated containers in air storage.

Yield and fruit size:

Mean fruit number.—389.

Mean yield.—6.1 kg.

Proportion of fruit <6 g.—2%.

Proportion of marked fruit.—38%. It is anticipated mature, well-managed vines would yield approximately 3000 fruit per vine with a mean fruit weight of 14 g and mean yield per vine of 42 kg.

Geographical adaptation: Trials indicate that the variety is well-suited to production in the warm-temperate Bay of Plenty region but also performs well in the more temperate region of Nelson, New Zealand.

1. A new and distinct kiwi plant of the species A. arguta substantially as herein described and illustrated, characterised by its green hairless, edible skin with red-centered flesh at maturity, small fruit size, oblong shape, and sweet aromatic taste.
